(19) United States
(12) Patent Application Publication
(10) Pub. No.: US 2016/0047786 A1
(43) Pub. Date: Feb. 18, 2016

(54) Food Storage Appliance with Moisture Sensor

(71) Applicant: Sunbeam Products, Inc., Boca Raton, FL (US)

(72) Inventor: David Owens, Boynton Beach, FL (US)

(73) Assignee: SUNBEAM PRODUCTS, INC., Boca Raton, FL (US)

(21) Appl. No.: 14/824,790

(22) Filed: Aug. 12, 2015

Related U.S. Application Data
(60) Provisional application No. 62/036,384, filed on Aug. 12, 2014.

(51) Int. Cl. G01N 33/02; G01N 27/04; G01N 27/02; A23L 3/015

(52) U.S. Cl. CPC G01N 33/02; A23L 3/0155; G01N 27/04; G01N 27/028; A23V 2002/00

(57) ABSTRACT

A food storage appliance including a housing, an elongated vacuum trough disposed in the housing, one or more electronic components including a vacuum motor fluidly connected to the vacuum trough and a heat sealing element disposed in proximity of the vacuum trough, an sensor disposed on the housing in proximity to the vacuum trough configured to generate an electrical signal if liquid is detected in a food storage container, the food storage container having a portion inserted into the vacuum trough prior to an evacuation operation, the sensor detecting liquid before it is drawn into the vacuum trough during the evacuation operation, and an integrated electronic circuit connected to the sensor configured to receive the electrical signal and configured to control one of the vacuum motor or the heat sealing element when liquid is detected in the food storage container.

### CROSS-REFERENCE TO RELATED APPLICATION

The present application claims the benefit of U.S. Provisional Patent Application No. 62/036,384 filed Aug. 12, 2014, entitled “Food Storage Appliance Moisture Sensor Solution”.

### FIELD OF THE INVENTION

The invention relates to food preservation, and more particularly to an improved food storage appliance having a sensor to detect liquid in a food storage container before the liquid is drawn into the vacuum trough.

### BACKGROUND OF THE INVENTION

Vacuum packaging appliances that evacuate air from containers holding food are becoming increasingly popular with households for food preservation and storage. The removal of the air delays spoilage and extends the life of the food. The appliances are typically used in conjunction with bag material that constitutes the container holding the food. After the food is inserted in the storage bag, the storage bag is fully sealed by applying heat and pressure to the remaining cut edges. A vacuum may be applied to evacuate air from the storage bag before it is fully sealed. Liquid in the storage bag is typically drawn into the appliance and may be directed into a cavity or a tray for discarding later.

### SUMMARY OF INVENTION

In an embodiment, there is provided a food storage appliance including a housing, an elongated vacuum trough disposed in the housing, one or more electronic components including a vacuum motor fluidly connected to the vacuum trough and a heat sealing element disposed in proximity of the vacuum trough, an sensor disposed on the housing in proximity to the vacuum trough configured to generate an electrical signal if liquid is detected in a food storage container, the food storage container having a portion inserted into the vacuum trough prior to an evacuation operation, the sensor detecting liquid before it is drawn into the vacuum trough during the evacuation operation, and an integrated electronic circuit connected to the sensor configured to receive the electrical signal and configured to control one of the vacuum motor or the heat sealing element when liquid is detected in the food storage container.

In an embodiment, there is provided a sensor for a food preservation appliance for detecting liquid in a food preservation container being processed by the food preservation appliance including a substrate comprised of a non-conducting material, a first trace comprised of a conducting material etched onto to the substrate having a plurality of first appendages extending perpendicularly therefrom and spaced along a lineal length of the first trace, a second trace comprised of a conducting material etched onto the substrate separated from the first trace by a first space, the second trace having a plurality of second appendages extending perpendicularly therefrom and spaced along a lineal length of the second trace, the second plurality of appendages interleaved with and separated by a second space from the first plurality of appendages, and a logic circuit electrically connected to the first and second traces, the logic circuit providing a first signal to the first trace and receiving a second signal from the second trace when liquid being evacuated from within the food preservation container by the food preservation appliance is disposed in proximity to the first and second spaces.

In an embodiment, there is provided a method of storing and preserving food in a food storage container using a food storage including the steps of: inserting a portion of the food storage container into a vacuum trough of the food storage machine, commencing evacuating the food storage container using a vacuum motor disposed in the food storage machine fluidly connected to the vacuum trough, using a sensor to generate a signal if liquid is detected in the food storage container prior to the liquid being drawn into the vacuum trough, using a logic circuit to compare the generated signal to a threshold signal, and using the logic circuit to control one or both of the vacuum motor evacuating the food container or a heat sealing element disposed in proximity of the vacuum trough to heat seal the food storage container.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a perspective view of an embodiment of a food storage appliance;

FIG. 2 is an elevated front perspective view of the food storage appliance of FIG. 1;

FIG. 3 is a block diagram of the major electronic components of the food storage appliance of FIG. 1;

FIG. 4 is an illustration showing the intended use of the food storage appliance of FIG. 1;

FIG. 5 is an illustration of a sensor for detecting liquids for use with the food storage appliance of FIG. 1; and

FIG. 6 is an illustration of the operation of the sensor of FIG. 5.

### DETAILED DESCRIPTION OF THE INVENTION

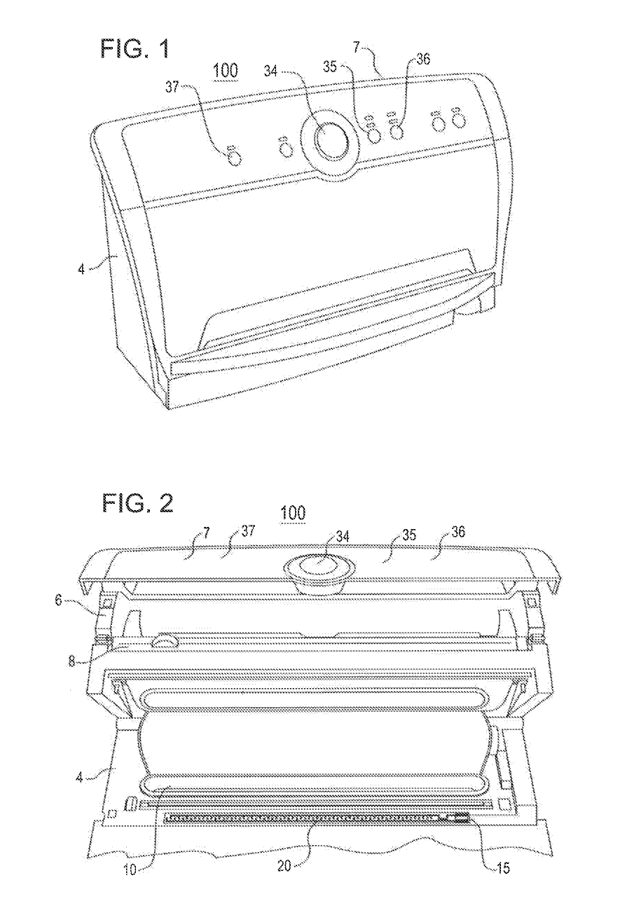

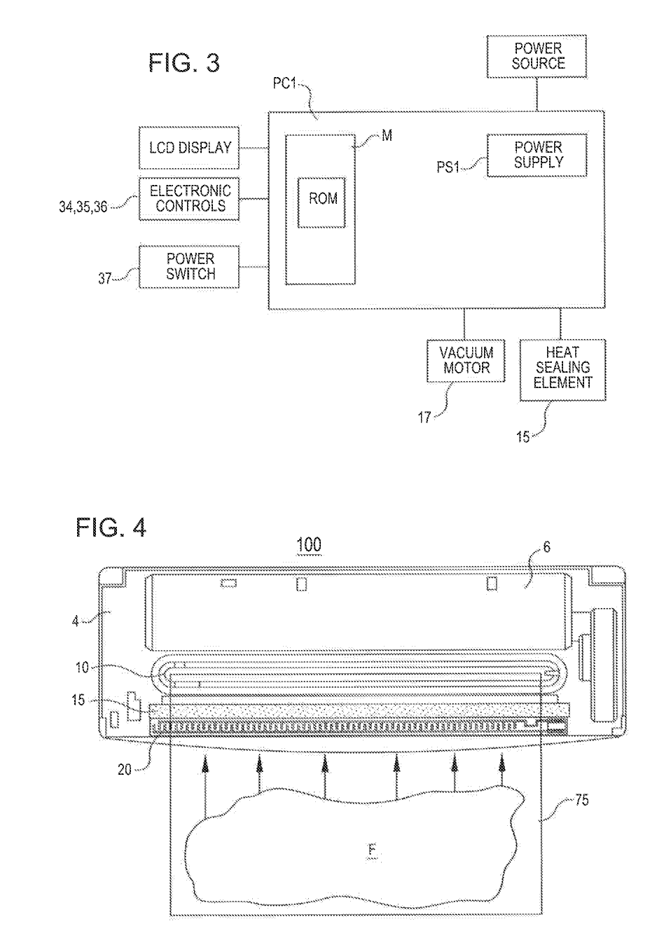

Referring now to FIGS. 1-4 of the drawings, there is illustrated an embodiment of a food storage appliance 100 which includes a housing 4, a packaging film compartment 6, a lid 7, a cutting mechanism 8, an elongated vacuum trough 10, an elongated heat sealing element 15 and a vacuum motor 17. The housing 4 encloses the packaging film compartment 6 covered by the lid 7 and the cutting mechanism 8 (inside the compartment 6) for cutting a length of packaging film stored on a roll in the packaging film compartment 6. The housing 4 includes a vacuum compartment including an elongated vacuum trough 10 for receiving an unsealed end of a food packaging container 75 (see FIG. 4) formed from a length of packaging film cut from the roll of packaging film. The heat sealing element 15 may be disposed on the housing 4 in front of the vacuum trough 10. An elongated sensor 20 is disposed in front of the heat sealing element 15 for detecting moisture or liquid in the food packaging container 75 when one end us inserted into the vacuum trough 10 during an evacuation and sealing operation. The sensor 20 generates an electrical signal when moisture or liquid is detected in the food packaging container 75 for controlling the operation of the food storage appliance 100 as described hereinbelow.

In an embodiment, the food preservation containers 75 may be pre-formed and sealed on three edges at the factory. Alternately, food packaging containers 75 may be pre-sealed along two edges at the factory and formed into a roll and stored in the packaging film compartment 6 as described for dispensing a length at a time. A third edge may be sealed by the user after being cut from the roll by inserting into the vacuum trough 10 and energizing the heat sealing element 15. In either scenario, a fully formed food packaging container 75 containing a food item F may be evacuated and sealed on the remaining unsealed end by inserting it into the vacuum trough 10 and energizing the vacuum motor 17 and/or the heat sealing element 15 via electronic controls on the housing 4 as described hereinbelow.

In particular, the electronic controls includes a user interface for controlling various functions of the food storage appliance 100. The electronic controls may include exteriorly exposed buttons (switches) 34, 35, 36, and 37 for access by the user. For example, the button 34 may operate both the vacuum motor 17 and the heat sealing element 15. Alternately, the button 35 may operate the heat sealing element 15 only. Alternately, the button 36 may operate the vacuum motor 17 only. Finally, the button 37 may control electrical power being provided to the electronic controls and the electronic components within housing 4 as described below.

Within the housing 4, the electronic components may include a microprocessor M mounted on a printed circuit board PC1 with an operating control program stored in ROM that controls the vacuum motor 17 and the heat sealing element 15, as discussed herein. The electronic components may also include other conventional components such as a power circuit PS1, an input interface circuit (not shown), an output interface circuit (not shown), and one or more storage devices ME, such as a ROM (Read Only Memory) device and a RAM (Random Access Memory) device. The power circuit PS1 is connected to an AC or DC power source and directs power to the motors, switches, sensors, etc. described herein, as well as provide power to other circuits and components of the electronic controls. The input interface circuit can be electrically connected to the buttons 34, 35, 36 and 37 for user control. The output interface circuit can be electrically connected to a LCD screen. The storage device ME stores processing results and control programs that are run by the microprocessor circuit M. The electronic controls are capable of selectively controlling any of the vacuum motor 17 and the heat sealing element 10 in accordance with the control program. It will be apparent to those skilled in the art from this disclosure that the precise structure and algorithms for the control panel can be any combination of hardware and software that will carry out the functions of the present invention.

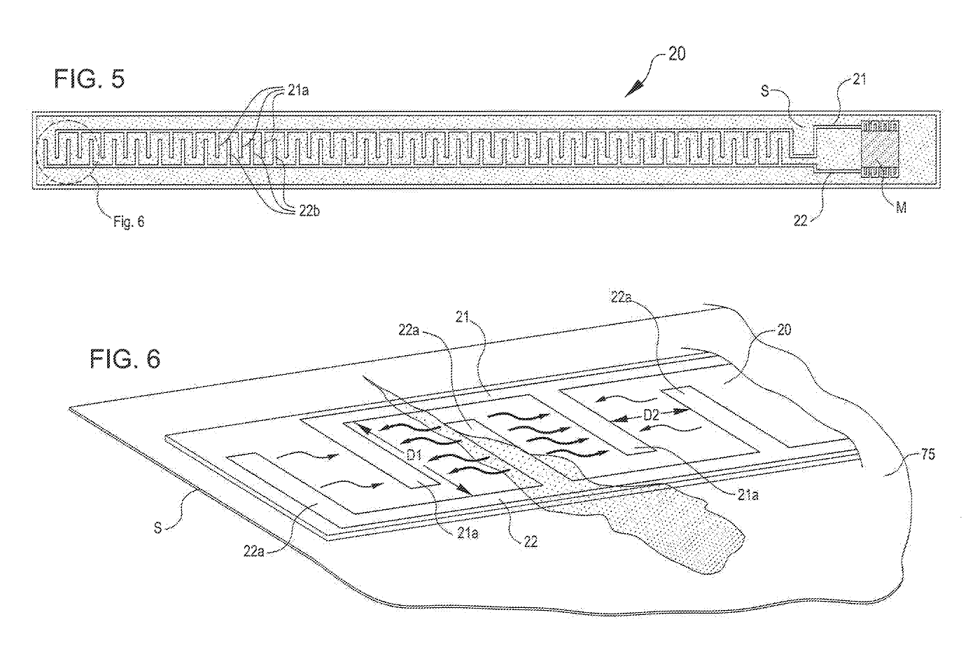

Referring now to FIGS. 5 and 6, there is illustrated an embodiment of a liquid or moisture sensor 20 that is electrically connected to a logic circuit or an integrated circuit such as the microprocessor M described above. The sensor 20 includes two elongated electrically conductive traces 21, 22 disposed adjacent one another etched onto a non-conductive substrate S. The traces 21, 22 may be comprised of a conductive material such as metal including but not limited to copper, gold, silver and aluminum. The traces 21, 22 are spaced apart from one another a small distance D1 forming a dielectric barrier therebetween. One of the traces 21 is a transmitting antenna fed a signal by the microprocessor M. The other trace 22 is a receiving antenna that receives electromagnetic signals generated by trace 21 and feeds the received signal back to the microprocessor M. Each of the traces 21, 22 have discrete appendages 21a, 22a extending perpendicularly therefrom along their length at equally spaced intervals such that the appendages 21a, 22a interleave with one another but spaced at a small distance D2 apart forming a dielectric barrier.

In an embodiment, normally when electrical power to the food storage machine 100 is turned on at button 37, the microprocessor M generates no signal or a weak signal provided to trace 21 and appendages 21a. As such, no signal or a weak signal is transmitted by the trace 21 and appendages 21a and no signal or a very weak signal is received by the trace 22 and appendages 22a. This signal or current in trace 21 and appendages 21a generates a corresponding electric field in the vicinity thereof. Still, because of the dielectric barrier between trace 21 and appendages 21a and trace 22 and appendages 22a, no signal or a very weak signal is received by trace 22 and appendages 22a.

If any liquid present in the food preservation container 75 during the evacuation operation is drawn towards the vacuum trough 10, it must pass over the traces 21, 22 and appendages 21a and 22a. The presence of the liquid in the spaces between the traces 21, 22 and appendages 21a and 22a changes the permittivity of air in the spaces. This reduced permittivity allows the electric field created by the weak signal provided to trace 21 and appendages 21a to induce a current or increases signal strength in trace 22 and appendages 22a. This induced current or received signal is provided to the microprocessor M which compares the signal to a threshold signal level. If the received signal is equal to or greater than the threshold signal level, this means that there is liquid in the food preservation container 75 about to he drawn into the vacuum trough 10. The microprocessor M may use this signal to modify control programs for the vacuum motor 17 and the heat sealing element 15. For example, upon detecting the presence of liquid in the food preservation container 75 the microprocessor M may automatically extend the amount of time the heat sealing element 15 is energized to improve seal quality. This minimizes or eliminates the need for a liquid drip tray in the vacuum trough 10. Alternately, the microprocessor M may shut off or slow the vacuum motor 17 so liquid is drawn more slowly or prevented from being drawn from the food preservation container 75 into the vacuum trough. The foregoing examples are not meant to be limiting as other possible modifications to the evacuating and sealing operations are possible.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims. A similar or nearly identical food preservation appliance is disclosed in U.S. patent application Ser. No. 14/126,692 filed Dec. 16, 2013, owned by a common assignee and incorporated by reference as if fully re-written herein.

What is claimed is:

1. A food storage appliance, comprising:
a housing;
an elongated vacuum trough disposed in the housing;
one or more electronic components including a vacuum motor fluidly connected to the vacuum trough and a heat sealing element disposed in proximity of the vacuum trough;
a sensor disposed on the housing in proximity to the vacuum trough configured to generate an electrical signal if liquid is detected in a food storage container, the food storage container having a portion inserted into the vacuum trough prior to an evacuation operation, the sensor detecting liquid before it is drawn into the vacuum trough during the evacuation operation; and
an integrated electronic circuit connected to the sensor configured to receive the electrical signal and configured to control one of the vacuum motor or the heat sealing element when liquid is detected in the food storage container.

2. The food storage appliance of claim 1, further including the integrated electronic circuit being a microprocessor.

3. The food storage appliance of claim 1, the sensor further including:
an elongated non-conducting substrate having an elongated first electrical trace having a first plurality of appendages formed thereon operating as a transmitting antenna spaced apart from an elongated second electrical trace formed thereon with a second plurality of appendages operating as a receiving antenna interleaved with and spaced apart from the first plurality of appendages,
wherein the first electrical trace and the first plurality of appendages transmits an electrical signal that is received by the second electrical trace and the second plurality of appendages if liquid in the food storage container is brought into the space between the first plurality of appendages and the second plurality of appendages during the evacuation operation.

4. The food storage appliance of claim 3, wherein a time the heat sealing element is energized for heat sealing is controlled by the integrated electronic circuit and dependent upon whether the second electrical trace and the second plurality of appendages receive the electrical signal above a threshold signal strength from the first electrical trace and the first plurality of appendages.

5. A sensor for a food preservation appliance for detecting liquid in a food preservation container being processed by the food preservation appliance, comprising:
a substrate comprised of a non-conducting material;
a first trace comprised of a conducting material etched onto to the substrate having a plurality of first appendages extending perpendicularly therefrom and spaced along a lineal. length of the first trace;
a second trace comprised of a conducting material etched onto the substrate separated from the first trace by a first space, the second trace having a plurality of second appendages extending perpendicularly therefrom and spaced along a lineal length of the second trace, the second plurality of appendages interleaved with and separated by a second space from the first plurality of appendages; and
a logic circuit electrically connected to the first and second traces, the logic circuit providing a first signal to the first trace and receiving a second signal from the second trace when liquid being evacuated from within the food preservation container by the food preservation appliance is disposed in proximity to the first and second spaces.

6. The sensor of claim 5, wherein the first trace and the first plurality of appendages are a transmitting antenna.

7. The sensor of claim 5, wherein the second trace and the second plurality of appendages are a receiving antenna The sensor of claim 5, the first trace, the first plurality of appendages, the second trace and the second plurality of appendages being comprised of metal.

9. The sensor of claim 8, wherein the metal is selected from the group consisting of copper, silver, aluminum and gold.

10. The sensor of claim 5, wherein the logic circuit is an integrated circuit which compares the first signal with the second signal and based on a determined differential controls processing of the food preservation container by the food preservation appliance.

11. The sensor of claim 5, wherein the logic circuit is a microprocessor which compares the first signal with the second signal and if the second signal meets a threshold signal strength generates a control signal to control processing of the food preservation container by the food preservation appliance.

12. A method of storing and preserving food in a food storage container using a food storage machine, comprising:
inserting a portion of the food storage container into a vacuum trough of the food storage machine;
commence evacuating the food storage container using a vacuum motor disposed in the food storage machine fluidly connected to the vacuum trough;
using a sensor to generate a signal if liquid is detected in the food storage container prior to the liquid being drawn into the vacuum trough;
using a logic circuit to compare the generated signal to a threshold signal; and
using the logic circuit to control one or both of the vacuum motor evacuating the food container or a heat sealing element disposed in proximity of the vacuum trough to heat seal the food storage container if the generated signal is above the threshold signal.

13. The method of claim 12, further including selecting the sensor to include:
a substrate comprised of a non-conducting material;
a first trace comprised of a conducting material etched onto the substrate having a plurality of first appendages extending perpendicularly therefrom and spaced along a lineal length of the first trace; and
a second trace comprised of a conducting material etched onto the substrate separated from the first trace by a first space, the second trace having a plurality of second appendages extending perpendicularly therefrom and spaced along a lineal length of the second trace, the second plurality of appendages interleaved with and separated by a second space from the first plurality of appendages.

14. The method of claim 12, further including selecting the logic circuit to be an integrated circuit or a microprocessor.

15. The method of claim 13, whereby the first trace and the plurality of first appendages are a transmitting antenna.

16. The method of claim 13, whereby the second trace and the plurality of second appendages are a receiving antenna.

17. The method of claim 13, further including selecting the conducting material from the group consisting of copper, silver, aluminum and gold.
